(19) United States
(12) Patent Application Publication
(10) Pub. No.: US 2025/0302280 A1
(43) Pub. Date: Oct. 2, 2025

(54) ENDOSCOPE

(71) Applicant: FUJIFILM Corporation, Tokyo (JP)

(72) Inventors: Naoki SUGIURA, Kanagawa (JP); Takashi HARADA, Kanagawa (JP)

(73) Assignee: FUJIFILM Corporation, Tokyo (JP)

(21) Appl. No.: 19/075,710

(22) Filed: Mar. 10, 2025

(30) Foreign Application Priority Data
Mar. 29, 2024 (JP) 2024-055394

(51) Int. Cl. A61B 1/00

(52) U.S. Cl. CPC A61B 1/00137; A61B 1/00177

(57) ABSTRACT

Provided is an endoscope in which an insulating property of a distal end member can be ensured. An endoscope includes a distal end member that is connected to a distal end side of an insertion part and that is made of a conductive material, and a distal end cap that is mounted on the distal end member, in which the distal end member has a disposition surface on which an observation window and an illumination window are disposed, and the endoscope includes a cover member that is provided on the disposition surface, that has a first insertion hole in which the observation window is disposed and a second insertion hole in which the illumination window is disposed, and that has an insulating property.

### CROSS-REFERENCE TO RELATED APPLICATION

The present application claims priority under 35 U.S.C § 119(a) to Japanese Patent Application No. 2024-055394 filed on Mar. 29, 2024, which is hereby expressly incorporated by reference, in its entirety, into the present application.

### BACKGROUND OF THE INVENTION

### 1. Field of the Invention

The present invention relates to an endoscope, and more particularly, to an endoscope including a distal end member in which an observation window and an illumination window are disposed.

### 2. Description of the Related Art

In an endoscope, various treatment tools are introduced from a treatment tool inlet port provided in an operating part, and the treatment tool is led out from a treatment tool outlet port that is open at a distal end portion of an insertion part to the outside and used for a treatment. For example, in a duodenoscope, a treatment tool, such as a guide wire, a contrast tube, or a high-frequency treatment tool (papillotomy knife), is used. Such a treatment tool needs to change a lead-out direction at the distal end portion in order to treat and examine a desired position in a subject. Therefore, a treatment tool elevator for elevating the treatment tool and the guide wire is provided in an elevator housing space formed in the distal end portion.

In a case in which the treatment is performed by using the high-frequency treatment tool, a spark current may be generated between the high-frequency treatment tool and the distal end member, and it is necessary to prevent the spark current from being generated even in a case in which the high-frequency treatment tool is used, for example.

Therefore, in JP2018-171256A, a distal end cap made of a resin is attachably and detachably mounted on a distal end member made of a metal in a duodenoscope. In JP2007-330756A, an abutment portion on which a guide wire abuts is formed by an insulating member, and a distal end cover (distal end cap) that covers a periphery of a distal end member made of a metal is made of a non-conductive material such as a resin. In JP1997-299316A (JP-H9-299316), an outer surface of a distal end member on a base end portion side is formed by an insulating portion of an insulating material, and, in a case in which a distal end cover having an insulating property is mounted on the distal end member, an inner peripheral surface and an outer surface of the distal end cover are in close contact with each other over the entire circumference.

### SUMMARY OF THE INVENTION

However, since a surface of the distal end member on which the illumination window and the observation window are disposed is exposed from a distal end cap, a spark current may be generated from the high-frequency treatment tool to the distal end member, and the endoscope may be damaged.

The present invention has been made in view of the above-described circumstances, and an object of the present invention is to provide an endoscope in which an insulating property of a distal end member can be ensured.

A first aspect relates to an endoscope comprising: a distal end member that is connected to a distal end side of an insertion part and that is made of a conductive material; and a distal end cap that is mounted on the distal end member, in which the distal end member has a disposition surface on which an observation window and an illumination window are disposed, and the endoscope includes a cover member that is provided on the disposition surface, that has a first insertion hole in which the observation window is disposed and a second insertion hole in which the illumination window is disposed, and that has an insulating property.

A second aspect relates to the endoscope according to the first aspect, in which the disposition surface has a recessed portion in which the cover member is disposed.

A third aspect relates to the endoscope according to the second aspect, in which the recessed portion is provided with an engaging portion, and the cover member has an engaged portion that engages with the engaging portion.

A fourth aspect relates to the endoscope according to the third aspect, in which the engaging portion is an engaging hole that is provided in a bottom surface portion of the recessed portion, and the engaged portion is a protrusion portion that is provided on the cover member and that is engageable with the engaging hole.

A fifth aspect relates to the endoscope according to the third or fourth aspect, further comprising: a retaining portion that prevents the engaged portion from being disengaged from the engaging portion.

A sixth aspect relates to the endoscope according to any one of the second to fifth aspects, in which the recessed portion has a flange portion that supports the illumination window, and the illumination window is fixed by the cover member and the flange portion.

A seventh aspect relates to the endoscope according to the sixth aspect, in which the recessed portion has a cylindrical portion that protrudes from a bottom surface portion of the recessed portion, and the flange portion is located inside the cylindrical portion.

An eighth aspect relates to the endoscope according to any one of the first to seventh aspects, in which an inner diameter of the first insertion hole as viewed from an optical axis direction of the observation window is equal to or larger than an outer diameter of the observation window.

A ninth aspect relates to the endoscope according to any one of the first to eighth aspects, in which the cover member has a first cover surface on which the first insertion hole is formed and a second cover surface on which the second insertion hole is formed, and the second cover surface has a higher height in a normal direction of the disposition surface than the first cover surface.

A tenth aspect relates to the endoscope according to the ninth aspect, in which the cover member has an inclined surface between the first cover surface and the second cover surface.

An eleventh aspect relates to the endoscope according to the ninth or tenth aspect, in which a normal line of the first cover surface includes a component toward a base end side of a longitudinal axis of the insertion part.

A twelfth aspect relates to the endoscope according to any one of the first to eleventh aspects, in which an optical axis of the observation window includes a component toward a base end side of a longitudinal axis of the insertion part.

A thirteenth aspect relates to the endoscope according to any one of the first to twelfth aspects, in which the cover member has a covered portion that is covered with the distal end cap in a case in which the distal end cap is mounted on the distal end member.

A fourteenth aspect relates to the endoscope according to any one of the first to thirteenth aspects, in which the distal end member is a side-viewing type in which the observation window and the illumination window face in a direction intersecting a longitudinal axis direction of the insertion part.

According to the aspects of the present invention, it is possible to ensure the insulating property of the distal end member.

### BRIEF DESCRIPTION OF THE DRAWINGS

FIG. 1 is an explanatory view showing an overall configuration of an endoscope.

FIG. 2 is an enlarged perspective view showing a case in which a distal end portion is viewed from a Z(+) direction side.

FIG. 3 is a perspective view showing a state in which a distal end cap is removed from a distal end member of FIG. 2.

FIG. 4 is a perspective view showing the distal end member as viewed from a Y(+)

FIG. 5 is a perspective view showing a state in which a cover member is removed from the distal end member.

FIGS. 6A to 6C are views showing the cover member as viewed from the Z(+) direction side, an X(−) direction side, and a Z(−) direction side.

FIG. 7 is an enlarged view showing a recessed portion.

FIG. 8 is a view showing retention of the cover member and the distal end member.

FIG. 9 is a view showing a state in which the distal end cap is mounted.

FIG. 10 is a cross-sectional view taken along X-X in FIG. 9.

### DESCRIPTION OF THE PREFERRED EMBODIMENTS

Hereinafter, an endoscope according to an embodiment of the present invention will be described with reference to the accompanying drawings.

### Overall Configuration of Endoscope 1

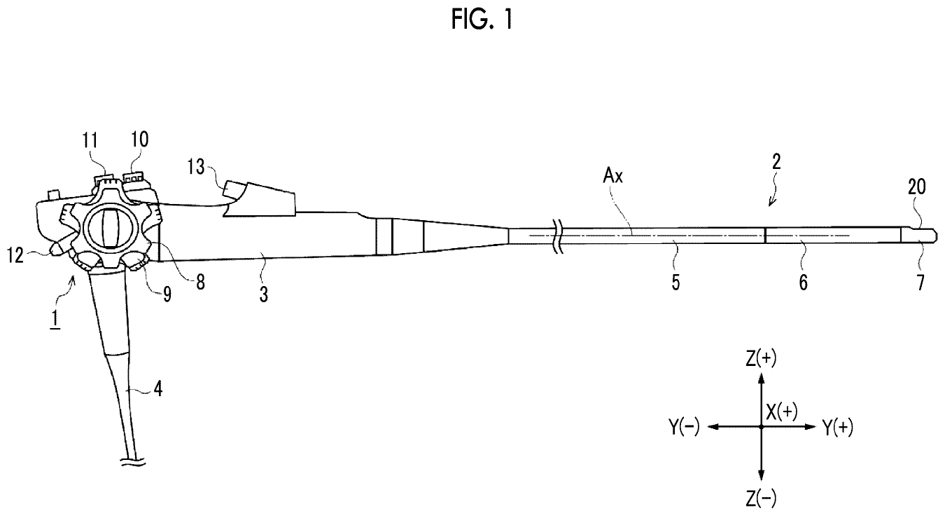

FIG. 1 is an overall configuration view showing an endoscope 1 according to the present embodiment.

As shown in FIG. 1, the endoscope 1 comprises an insertion part 2 that is inserted into a subject, and an operating part 3 that is connected to a base end portion of the insertion part 2 and is operated by an operator. It should be noted that, in the embodiment, an endoscope used as a duodenoscope is described as an example.

Hereinafter, in a case of describing a configuration of each part of the endoscope 1, a three-dimensional orthogonal coordinate system of X, Y, and Z will be used for convenience of description. A Z direction in the drawings is an up-down direction, a Z(+) direction side indicates an up direction, and a Z(−) direction side indicates a down direction. In addition, an X direction in the drawings is a direction perpendicular to the Z direction, an X(+) direction side indicates a right direction, and an X(−) direction side indicates a left direction. In addition, a Y direction in the drawings is a direction perpendicular to both the Z direction and the X direction, a Y(+) direction side indicates a distal end side direction, and a Y(−) direction side indicates a base end side direction. It should be noted that, in addition, each of the above-described directions refers to a direction in a case in which the operator views the insertion part 2 from an upper side in a case in which the operator grips the operating part 3 of the endoscope 1.

### Insertion Part 2

The insertion part 2 of the endoscope 1 is inserted into the subject through an oral cavity and is further inserted into a duodenum from an esophagus via a stomach. Accordingly, a procedure such as an examination or a treatment of the duodenum is performed by using a treatment tool inserted into the insertion part 2.

Examples of the treatment tool include biopsy forceps having a cup that can collect a biological tissue at a distal end portion thereof, an endoscopic sphincterotomy (EST) knife, a contrast tube, a high-frequency treatment tool (papillotomy knife), and a guide wire.

The insertion part 2 has a longitudinal axis Ax, and has a soft portion 5, a bendable portion 6, and a distal end portion 7 in order from a base end side to a distal end side. It should be noted that a longitudinal axis Ax direction and the Y direction are the same direction.

The soft portion 5 occupies most of the insertion part 2 from the base end side, and is formed by, for example, a metal spiral tube and a net (not shown), and is further formed by a resin tube covering the outer periphery of the metal spiral tube and the net. The soft portion 5 has flexibility that allows the soft portion 5 to be bent in any direction, and is bent along an insertion path into a body cavity in a case in which the insertion part 2 is inserted into the body cavity.

The bendable portion 6 has a tubular body in which a plurality of angle rings are connected to each other in a swingable manner, and is formed by covering an outer periphery of the tubular body with a mesh body woven with a metal wire and further covering the outer periphery of the mesh body with angle rubber made of rubber. In addition, a plurality of wires extend from the angle knobs 8 and 9 of the operating part 3 to the bendable portion 6, and distal end portions of these wires are fixed to the distal end portion of the tubular body. Accordingly, the bendable portion 6 is bent in the up-down direction (Z direction) and the left-right direction (X direction) in response to a rotational movement operation of the angle knobs 8 and 9.

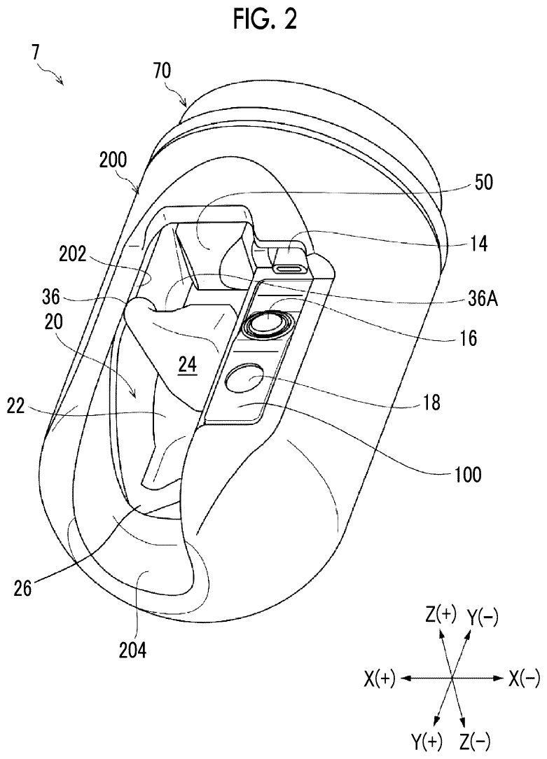

The distal end portion 7 has a distal end member 20 and a distal end cap 200 (see FIG. 2) that is attachably and detachably mounted on the distal end member 20.

The distal end member 20 is provided at the distal end portion 7, and is made of a metal material having corrosion resistance. An axial direction of the distal end member 20 is the same as the longitudinal axis Ax direction and the Y direction. The distal end cap 200 is made of an elastic material, for example, silicone rubber. The distal end member 20 and the distal end cap 200 are examples of a distal end member and a distal end cap according to the embodiment of the present invention, respectively.

### Operating Part 3

As shown in FIG. 1, the operating part 3 includes angle knobs 8 and 9 for bending the bendable portion 6, an air/water supply button 10, a suction button 11, an elevating operation lever 12, and a treatment tool inlet port 13.

The air/water supply button 10 is an operation button for performing air/water supply from an air/water supply nozzle 14 (see FIG. 2) of the distal end portion 7 toward an observation window 16, an illumination window 18, and the like via an air/water supply channel (not shown) inserted into the insertion part 2.

The suction button 11 is an operation button for suctioning a body fluid and the like via a treatment tool insertion channel (not shown) inserted into the insertion part 2.

The elevating operation lever 12 is a lever for performing an operation of elevating and lowering an elevator 36 (see FIG. 2) provided at the distal end portion 7. The elevating operation lever 12 is one of constituent members of a treatment tool elevating mechanism.

The treatment tool inlet port 13 communicates with the treatment tool insertion channel, and is an opening through which the operator introduces the treatment tool or the like. The treatment tool introduced from the treatment tool inlet port 13 is guided to the distal end portion 7 through the treatment tool insertion channel.

A universal cord 4 is connected to the operating part 3, and the universal cord 4 is connected to an image processing device and a light source device (not shown). A monitor device (not shown) is connected to the image processing device via a cable. The overall structure of the endoscope 1 is as described above. Hereinafter, a structure of the distal end portion 7 will be described.

### Distal End Portion 7

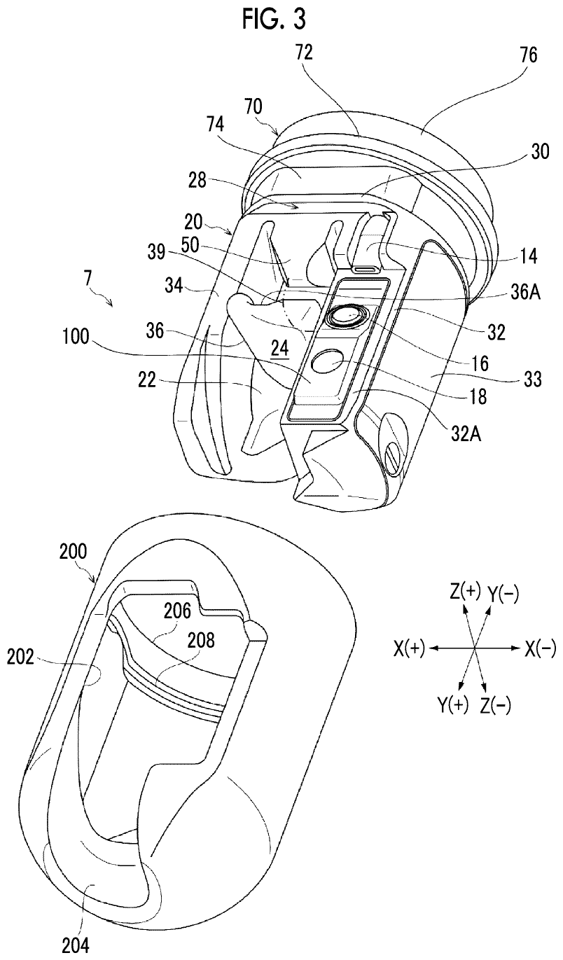

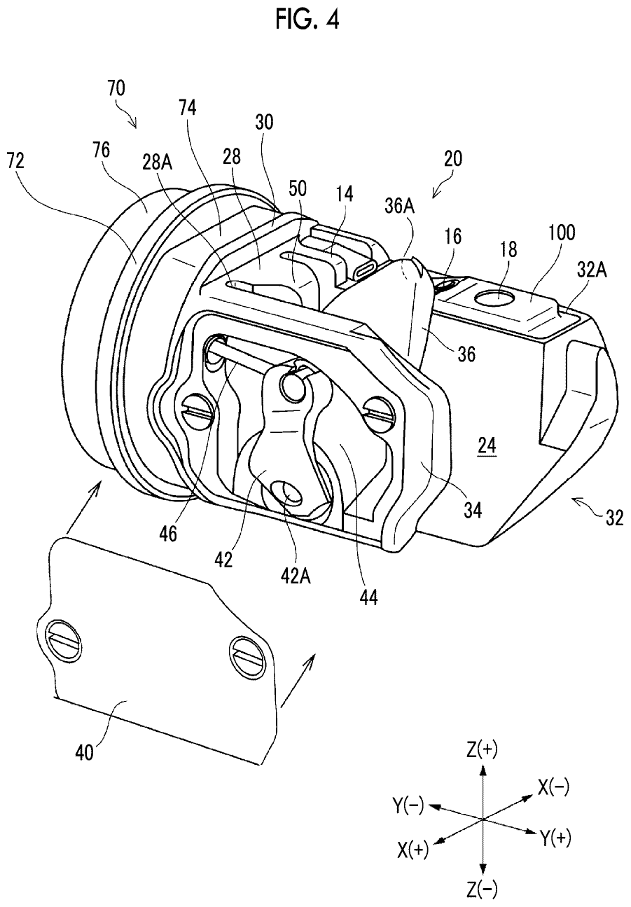

FIG. 2 is an enlarged perspective view showing a case in which the distal end portion 7 is viewed from the Z(+) direction side. FIG. 2 shows a state in which the distal end cap 200 is mounted on the distal end member 20. In addition, FIG. 3 is a perspective view showing a state in which the distal end cap 200 is removed from the distal end member 20 of FIG. 2. FIG. 4 is a perspective view showing the distal end portion 7 as viewed from the Y(+) direction side.

As shown in FIG. 2, in a state in which the distal end cap 200 is mounted on the distal end member 20, the distal end cap 200 has an upper opening 202 that communicates with an opening 24 on the Z(+) direction side of an elevator housing portion 22 to be described later.

Subsequently, a configuration of the distal end cap 200 will be briefly described.

As shown in FIG. 3, the distal end cap 200 is formed in a substantially tubular shape to house the distal end member 20. In addition, the distal end cap 200 has an upper opening 202 that communicates with the opening 24 of the elevator housing portion 22, a distal end portion opening 204 that communicates with a distal end opening 26 of the elevator housing portion 22 on the Y(+) direction side, and a base end portion opening 206 for mounting the distal end cap 200 on the distal end member 20. An engaging groove 208 of the distal end cap 200 is locked to a flange portion 30 that protrudes on an outer peripheral surface of a base portion 28 of the distal end member 20.

The upper opening 202 is provided on the Z(+) direction side, and is formed to have a size at which the distal end portion of the air/water supply nozzle 14, the observation window 16, the illumination window 18, and a guide wire fixing portion 50 described later are exposed in addition to the elevator housing portion 22 (opening 24). In addition, the distal end portion opening 204 is installed consecutively to the upper opening 202, and is formed as an opening for air venting for preventing a body cavity wall from being suctioned to the upper opening 202 during a suction operation of the endoscope 1. In addition, the base end portion opening 206 is an opening for inserting the distal end member 20.

Subsequently, a configuration of the distal end member 20 will be described.

As shown in FIG. 3, the distal end member 20 has a first wall portion 32 and a second wall portion 34 that face each other in the X direction. The first wall portion 32 is disposed on the X(−) direction side, and the second wall portion 34 is disposed on the X(+) direction side. The first wall portion 32 and the second wall portion 34 are made of a conductive material.

In addition, the first wall portion 32 and the second wall portion 34 are installed consecutively to a base portion 28 of the distal end member 20 at the respective base ends thereof, and extend from the base portion 28 to the Y(+) direction side.

Further, the elevator housing portion 22 for housing the elevator 36 is provided between the first wall portion 32 and the second wall portion 34 in the X direction. The elevator housing portion 22 is provided adjacent to the first wall portion 32 and the second wall portion 34, and is formed as a slit-shaped space along the Y direction.

The distal end member 20 is provided with a treatment tool outlet port 39 (indicated by a virtual line in FIG. 3). The distal end side of the treatment tool insertion channel communicates with the treatment tool outlet port 39. The treatment tool insertion channel is inserted into the insertion part 2 (see FIG. 1), and the base end side of the treatment tool insertion channel is connected to the treatment tool inlet port 13 of the operating part 3.

Therefore, the treatment tool introduced from the treatment tool inlet port 13 is guided to the elevator housing portion 22 through the treatment tool insertion channel and the treatment tool outlet port 39. It should be noted that the treatment tool guided to the elevator housing portion 22 is changed in a lead-out direction by the elevator 36.

### First Wall Portion 32

As shown in FIGS. 2 to 4, the observation window 16 and the illumination window 18 are provided on the first wall portion 32. In addition, an imaging unit and an illumination unit are provided in the first wall portion 32. The first wall portion 32 is sealed by a distal end member cover 33 such that the imaging unit and the illumination unit are not exposed to the outside. The observation window 16 and the illumination window 18 are examples of an observation window and an illumination window according to the embodiment of the present invention.

The first wall portion 32 comprises a disposition surface 32A for disposing the observation window 16 and the illumination window 18. The disposition surface 32A is formed by a surface parallel to the X-Y plane, and faces the Z(+) direction side. It should be noted that the disposition surface 32A need only be formed by at least a surface that faces the Z(+) direction side. That is, the disposition surface 32A need only be formed by a surface including a normal component toward the Z(+) direction side. For example, the disposition surface 32A may be a surface substantially parallel to the X-Y plane (surface slightly inclined with respect to the X-Y plane). The disposition surface 32A is formed as a substantially rectangular surface in which a length in the Y direction is longer than a length in the X direction in a case of being viewed from the Z(+) direction side. The disposition surface 32A is an example of a disposition surface according to the embodiment of the present invention.

The imaging unit has an imaging optical system disposed inside the observation window 16 and a complementary-metal-oxide semiconductor (CMOS) type or charge-coupled device (CCD) type imaging element. The imaging element is connected to an image processing device via a signal cable inserted into the insertion part 2. An imaging signal of a subject image obtained by the imaging unit is input to the image processing device through the signal cable, and is displayed as the subject image on the monitor connected to the image processing device through the cable.

The illumination unit includes an illumination lens installed inside the illumination window 18 and a light guide disposed such that a distal end thereof faces the illumination lens. The light guide is inserted into the insertion part 2, and a base end portion thereof is connected to the light source device. As a result, irradiation light from the light source device is transmitted through the light guide and is emitted from the illumination window 18.

The observation window 16 and the illumination window 18 are disposed in this order along the Y(+) direction from the base portion 28 on the disposition surface 32A. Each of the observation window 16 and the illumination window 18 faces the Z(+) direction side. That is, the endoscope 1 according to the embodiment is a side-viewing endoscope, and the observation window 16 and the illumination window 18 face a direction intersecting the Y direction (longitudinal axis Ax direction). It should be noted that a cover member 100, which will be described later, is provided on the disposition surface 32A.

### Second Wall Portion 34

As shown in FIG. 4, a lever housing chamber 44 that has a recessed shape and that houses a lever 42 is provided in the second wall portion 34. Among two wall surfaces of the second wall portion 34 facing each other in the X direction, the lever housing chamber 44 is provided on a wall surface on a side opposite to a wall surface that faces the elevator housing portion 22.

The lever 42 in the lever housing chamber 44 is provided with a rotation shaft 42A at one end portion thereof. The rotation shaft 42A penetrates the second wall portion 34 in the X direction, and is connected to the elevator 36. Further, the other end portion of the lever 42 is connected to an operating wire 46. The operating wire 46 is inserted into the insertion part 2 (see FIG. 1), and is connected to the elevating operation lever 12 of the operating part 3.

Therefore, the operator operates the elevating operation lever 12 to push or pull the operating wire 46, and the lever 42 swings in conjunction with the push-pull operation. Accordingly, the elevator 36 swings using the rotation shaft 42A as a swing shaft, and a position of the elevator 36 is changed between an elevated position and a fallen position. Here, the treatment tool elevating mechanism is formed by the lever 42 having the rotation shaft 42A, the operating wire 46, and the elevating operation lever 12. It should be noted that the lever housing chamber 44 is sealed by the cover 40.

### Elevator 36

As shown in FIGS. 2 to 4, the elevator 36 is provided in the distal end member 20, and is housed in the elevator housing portion 22. In addition, the elevator 36 is attached to the second wall portion 34 swingably via the rotation shaft 42A. The elevator 36 is driven by the treatment tool elevating mechanism to perform an elevating operation and a falling operation.

The elevator 36 has a guide surface 36A (see FIG. 2 and the like). The guide surface 36A faces the Z(+) side in a case in which the elevator 36 is located at the fallen position, and faces the guide wire fixing portion 50 side in a case in which the elevator 36 is located at the maximum elevated position. The guide surface 36A is a surface for guiding the treatment tool, and is formed in a substantially U-shape that is recessed inward from the base end side that is a swing end of the elevator 36 to the distal end side.

In a case in which the elevator 36 is elevated by the treatment tool elevating mechanism, the treatment tool guided to the elevator housing portion 22 is guided by the guide surface 36A of the elevator 36 in a direction toward the opening 24 of the elevator housing portion 22 and is led out from the upper opening 202 of the distal end cap 200 to the outside. In addition, the lead-out direction of the treatment tool can be changed by adjusting an elevating angle of the elevator 36 by the treatment tool elevating mechanism.

### Guide Wire Fixing Portion 50

As shown in FIGS. 2 to 4, the guide wire fixing portion 50 is provided at the base portion 28. The guide wire fixing portion 50 protrudes from a distal end wall portion 28A of the base portion 28 toward the elevator housing portion 22 on the Y(+) direction side.

The guide wire fixing portion 50 is formed as a tapered protruding portion in the Y(+) direction. In other words, the guide wire fixing portion 50 is formed to have a substantially trapezoidal cubic shape with the distal end wall portion 28A as a bottom surface.

In a case in which the elevator 36 is elevated to the maximum elevated position, a guide wire (not shown) guided from the treatment tool insertion channel to the elevator housing portion 22 is interposed and fixed between the protruding distal end portion of the guide wire fixing portion 50 and the guide surface 36A having a recessed shape of the elevator 36. It should be noted that the guide wire fixing portion 50 is installed adjacent to the air/water supply nozzle 14 in the X direction.

### Insulating Ring 70

As shown in FIGS. 3 and 4, an insulating ring 70 having an annular shape is mounted on the base portion 28 of the distal end member 20 from the base end side. The insulating ring 70 is made of an insulating material, such as plastic or ceramic.

The insulating ring 70 has a flange portion 72 that protrudes outward in the radial direction along an outer peripheral surface thereof, a cap mounting portion 74 that is disposed on the distal end side with respect to the flange portion 72, and a bendable portion mounting portion 76 that is disposed on the base end side with respect to the flange portion 72. The cap mounting portion 74 is mounted on the base portion 28, and the insulating ring 70 is mounted on the distal end member 20. In this case, the cap mounting portion 74 is disposed on the base end side of the flange portion 30.

As shown in FIG. 2, the distal end cap 200 is mounted on the distal end member 20 in a state in which the insulating ring 70 is mounted on the distal end member 20. In this case, the distal end cap 200 is externally mounted on the distal end member 20 from the distal end side of the distal end member 20 to the base end side, and the base end of the distal end cap 200 abuts on the flange portion 72 of the insulating ring 70.

As a result, the engaging groove 208 of the distal end cap 200 engages with the flange portion 30 of the distal end member 20, and the distal end cap 200 is externally mounted on the cap mounting portion 74 of the insulating ring 70. As a result, the distal end cap 200 is mounted on the distal end member 20, and the distal end member 20 made of a conductive material is covered with the distal end cap 200 and the insulating ring 70, and the insulating property for the distal end member 20 is ensured.

As shown in FIGS. 3 and 4, the bendable portion mounting portion 76 is formed in a cylindrical shape. The bendable portion mounting portion 76 is a portion on which a distal end portion of the angle rubber of the bendable portion 6 is mounted. The distal end portion of the angle rubber of the bendable portion 6 is mounted on the bendable portion mounting portion 76, and the distal end of the angle rubber abuts on the flange portion 72. As a result, the angle rubber can be mounted on the insulating ring 70. It should be noted that the angle rubber is fixed to the insulating ring 70 by winding a thread (not shown) around the distal end portion thereof. The thread is fixed to the angle rubber with an adhesive (not shown).

As described above, the insulating property is ensured by covering the distal end member 20 with the distal end cap 200 and the insulating ring 70. However, the distal end cap 200 has open portions corresponding to the observation window 16 and the illumination window 18. That is, since the disposition surface 32A of the distal end member 20 of the conductive member is exposed, it is necessary to prevent a spark current from being generated even in a case in which a high-frequency treatment tool is used, for example.

Therefore, in the present invention, the cover member 100 having the insulating property described later is disposed on the disposition surface 32A to ensure the insulating property of the distal end member 20. The cover member 100 is an example of a cover member according to the embodiment of the present invention.

Hereinafter, the cover member 100 will be described.

### Cover Member 100

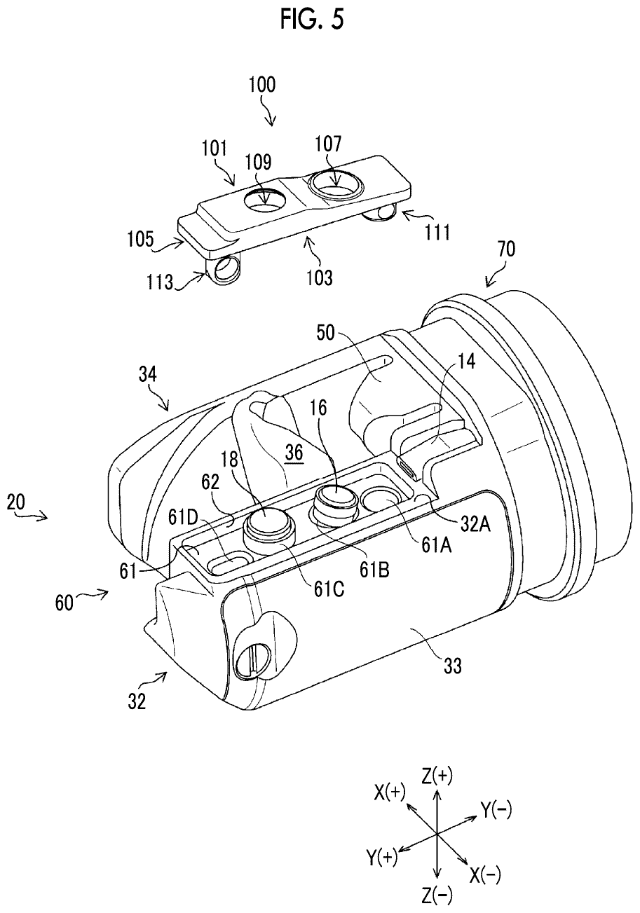

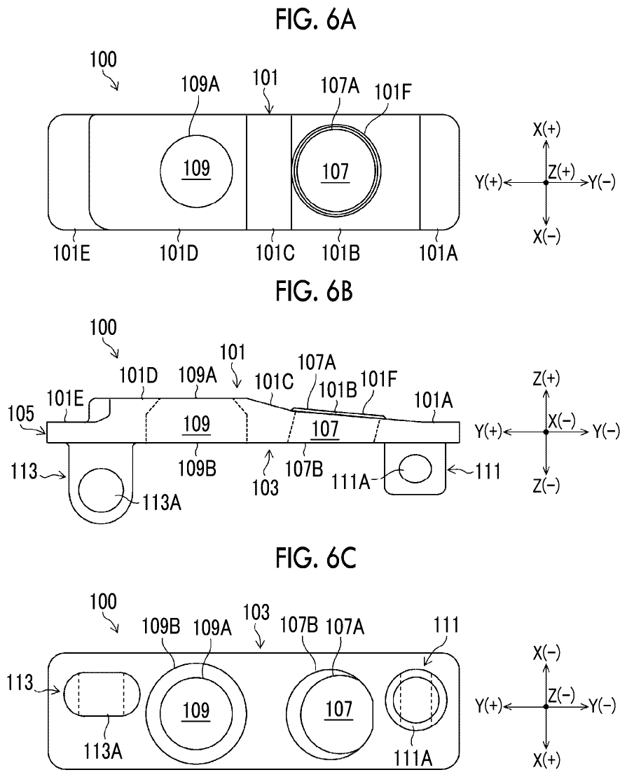

FIG. 5 is a perspective view showing a state in which the cover member 100 is removed from the distal end member 20. FIGS. 6A to 6C are views showing the cover member 100 as viewed in three directions. FIG. 6A is a view as viewed from the Z(+) direction side, FIG. 6B is a view as viewed from the X(−) direction side, and FIG. 6C is a view as viewed from the Z(−)

The cover member 100 is disposed on the disposition surface 32A of the first wall portion 32, and has an elongated flat plate shape as a whole. The cover member 100 is formed by an upper surface 101 facing the Z(+) direction side, a lower surface 103 facing the Z(−) direction side, and a side surface 105 connecting the upper surface 101 and the lower surface 103. The cover member 100 is provided with a first insertion hole 107 in which the observation window 16 is disposed, and a second insertion hole 109 in which the illumination window 18 is disposed. In addition, the cover member 100 is provided with a first protrusion portion 111 and a second protrusion portion 113, which will be described later.

As shown in FIG. 6A, in a case in which the cover member 100 is viewed in the Z(+) direction, the cover member 100 has a substantially rectangular shape in which the Y direction is the longitudinal direction. Hereinafter, each surface constituting the cover member 100 will be described.

The upper surface 101 has a plurality of surfaces different from each other. That is, the upper surface 101 has a base end side surface 101A, an observation window surface 101B, an intermediate surface 101C, an illumination window surface 101D, and a distal end side surface 101E in order from the Y(−) direction side to the Y(+) direction side (FIGS. 6A and 6B).

The base end side surface 101A is a surface located closest to the base end side (Y(−) direction side) in the Y direction, and is formed by a surface parallel to the X-Y plane. It should be noted that the term “parallel” used herein does not mean that the two lines are completely parallel to each other, and includes a case in which the two lines are substantially parallel to each other (the same applies hereinafter).

The observation window surface 101B is disposed at a position adjacent to the Y(+) direction side of the base end side surface 101A. The observation window surface 101B is a surface on which an opening 107A of the first insertion hole 107 is formed. It should be noted that the observation window 16 is disposed in the first insertion hole 107. The observation window surface 101B is formed by an inclined surface that is inclined with respect to the X-Y plane. Specifically, the normal line of the observation window surface 101B includes a normal component toward the Z(+) direction side and a normal component toward the Y(−) direction side. In other words, the normal line of the observation window surface 101B includes a component toward at least the base end side in the Y direction (longitudinal axis Ax direction). The observation window surface 101B is an example of a first cover surface according to the embodiment of the present invention.

The intermediate surface 101C is an example of an inclined surface according to the embodiment of the present invention, and is a surface connecting the observation window surface 101B and the illumination window surface 101D. The intermediate surface 101C is formed by an inclined surface that is inclined with respect to the X-Y plane, as in the observation window surface 101B, but has a different inclined angle (angle formed with the X-Y plane) from the observation window surface 101B. Specifically, the inclined angle of the intermediate surface 101C is larger than the inclined angle of the observation window surface 101B. It should be noted that, in the present embodiment, as a preferable aspect, a case has been described in which the inclined angle of the intermediate surface 101C is larger than the inclined angle of the observation window surface 101B, but the present invention is not limited to this, and the inclined angle of the intermediate surface 101C may be smaller than or the same as the inclined angle of the observation window surface 101B. In addition, the intermediate surface 101C may be formed by a surface having one or a plurality of steps.

The illumination window surface 101D is disposed at a position adjacent to the Y(+) direction side of the intermediate surface 101C. The illumination window surface 101D is a surface on which an opening 109A of the second insertion hole 109 is formed. It should be noted that the illumination window 18 is disposed in the second insertion hole 109. The illumination window surface 101D is formed by a surface parallel to the X-Y plane. That is, the illumination window surface 101D includes a normal component toward the Z(+) direction side. Regarding the position in the Z direction, the illumination window surface 101D is disposed on the Z(+) direction side as compared with the observation window surface 101B. That is, in a case in which the Z(+) direction side, which is a normal direction of the disposition surface 32A, is defined as a height direction, the illumination window surface 101D is disposed at a position having a higher height in the height direction (normal direction of the disposition surface 32A) than the observation window surface 101B. The illumination window surface 101D is an example of a second cover surface according to the embodiment of the present invention.

The distal end side surface 101E is a surface located on the most distal end side (Y(+) direction side) in the Y direction in the cover member 100. The distal end side surface 101E is formed by a surface parallel to the X-Y plane. The distal end side surface 101E is disposed at a position adjacent to the Y(+) direction side of the illumination window surface 101D via a stepped portion, and is disposed at a position having a lower height in the height direction (normal direction of the disposition surface 32A) than the illumination window surface 101D. The lower surface 103 is formed by a surface parallel to the X-Y plane (FIGS. 6B and 6C). An opening 107B of the first insertion hole 107 and an opening 109B of the second insertion hole 109 are provided on the lower surface 103. Further, a first protrusion portion 111 and a second protrusion portion 113 are provided on the lower surface 103. The first protrusion portion 111 and the second protrusion portion 113 will be described later.

The side surface 105 is a surface that connects the upper surface 101 and the lower surface 103, and is formed by a surface orthogonal to the X-Y plane.

As shown in FIG. 6B, the first insertion hole 107 is formed in a cylindrical shape, and an axial direction thereof is inclined to the Y(−) direction side with respect to the Z direction. The first insertion hole 107 formed as described above has the opening 107A on the upper surface 101 side and the opening 107B on the lower surface 103 side, and is formed to allow the observation window 16 to be inserted. The opening 107A and the opening 107B have a substantially circular shape. The center of the opening 107B is located on the Y(+) direction side with respect to the center of the opening 107A. A straight line (axis of the first insertion hole 107) connecting the center of the opening 107B and the center of the opening 107A is parallel to the normal line of the observation window surface 101B, is obliquely inclined with respect to the Z direction, and includes a component toward the base end side (Y(−) direction side) in the Y direction (longitudinal axis Ax). Further, a bank portion 101F is formed on a peripheral surface of the opening 107A on the upper surface 101 side.

In addition, the second insertion hole 109 is formed such that the lower surface 103 side has a cylindrical shape and the upper surface 101 side has a frustum shape, and the axial direction thereof is parallel to the Z direction. It should be noted that a truncated conical portion forming the second insertion hole 109 is formed in a tapered shape that is reduced in diameter toward the upper surface 101 side. The second insertion hole 109 formed as described above has the opening 109A on the upper surface 101 side and the opening 109B on the lower surface 103 side, and is formed to allow the illumination window 18 to be inserted. The opening 109A and the opening 109B have a substantially circular shape. Am inner diameter of the opening 109A is smaller than an inner diameter of the opening 109B. A straight line (axis of the second insertion hole 109) connecting the center of the opening 109A and the center of the opening 109B is parallel to the Z direction, and the opening 109A and the opening 109B are in a concentric circular positional relationship in a case of being viewed from the Z(−) direction side. It should be noted that the first insertion hole 107 and the second insertion hole 109 are examples of a first insertion hole and a second insertion hole according to the embodiment of the present invention.

As shown in FIGS. 6B and 6C, the first protrusion portion 111 is provided on the base end side of the cover member 100 and extends from the lower surface 103 to the Z(−) direction side. The first protrusion portion 111 has a substantially cylindrical outer shape. A first through-hole 111A extending in the X direction is formed in the first protrusion portion 111.

In addition, the second protrusion portion 113 is provided on the distal end side of the cover member 100, and extends to the Z(−) direction side from the lower surface 103. The second protrusion portion 113 has an elongated elliptical outer shape in which the Y direction is longer than the X direction in a case of being viewed from the Z(−) direction side. A second through-hole 113A extending in the X direction is formed in the second protrusion portion 113. It should be noted that each of the first protrusion portion 111 and the second protrusion portion 113 is disposed at a position offset to the X(−) direction side from the center position in the X direction.

### Recessed Portion 60

As shown in FIG. 5, a recessed portion 60 for disposing the cover member 100 is formed on the disposition surface 32A of the first wall portion 32. The recessed portion 60 is defined by a bottom surface portion 61 that is located on the Z(−) direction side with respect to the disposition surface 32A and a wall surface 62 that surrounds the bottom surface portion 61. The bottom surface portion 61 is formed by a surface parallel to the X-Y plane. The wall surface 62 is formed by a surface orthogonal to the X-Y plane. The recessed portion 60 and the bottom surface portion 61 are examples of a recessed portion and a bottom surface portion according to the embodiment of the present invention.

A shape of the bottom surface portion 61 is substantially the same as a shape of the lower surface 103 of the cover member 100 as viewed in the Z direction. The height of the wall surface 62 (distance in the Z direction between the bottom surface portion 61 and the disposition surface 32A) is substantially the same as the height of the side surface 105 corresponding to the base end side surface 101A and the distal end side surface 101E of the side surface 105 of the side surface 105 of the cover member 100. The term “substantially” includes a range that is allowed rather than a complete match. Therefore, in a case in which the cover member 100 is disposed in the recessed portion 60, the base end side surface 101A and the distal end side surface 101E are substantially flush with the disposition surface 32A.

A first engaging hole 61A and a second engaging hole 61D are provided on the bottom surface portion 61. In a case in which the cover member 100 is disposed in the recessed portion 60, the first engaging hole 61A is formed in a shape and a position where the first protrusion portion 111 of the cover member 100 can be engaged. Further, the second engaging hole 61D is formed in a shape and a position where the second protrusion portion 113 of the cover member 100 can be engaged.

In addition, an observation window hole 61B and a cylindrical portion 61C are provided on the bottom surface portion 61. The observation window hole 61B is a substantially circular hole portion, and the observation window 16 is led out from the observation window hole 61B. The cylindrical portion 61C has a substantially cylindrical shape protruding from the bottom surface portion 61 to the Z(+) direction side, and the illumination window 18 is disposed on the Z(+) direction side of the cylindrical portion 61C.

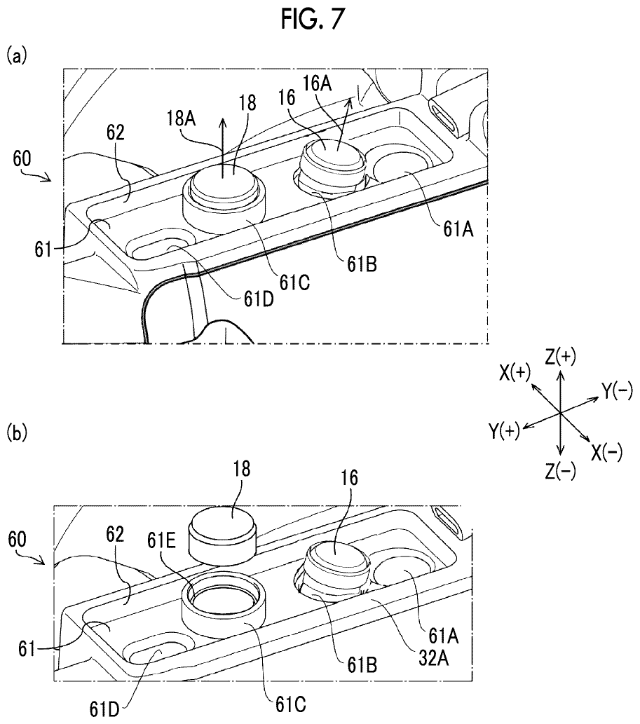

FIG. 7 is an enlarged view showing the recessed portion 60. Here, (a) of FIG. 7 is a view in which the illumination window 18 is disposed in the cylindrical portion 61C, and (b) of FIG. 7 is a view in which the illumination window 18 is removed from the cylindrical portion 61C in the Z(+) direction. As shown in (b) of FIG. 7, a flange portion 61E that supports the lower surface (surface on the Z(−) direction side) of the illumination window 18 is provided on an inner surface of the cylindrical portion 61C. The flange portion 61E is provided over the entire inner surface, but the flange portion 61E may be provided on a part or a plurality of parts of an entire inner surface of the cylindrical portion 61C as long as the illumination window 18 can be supported from the lower surface.

Subsequently, directions of an optical axis 16A of the observation window 16 and an optical axis 18A of the illumination window 18 will be described. As shown in (a) of FIG. 7, the optical axis 16A of the observation window 16 is inclined with respect to the X-Y plane. Specifically, the optical axis 16A includes a component toward the Z(+) direction side and a component toward the Y(−) direction side. That is, the optical axis 16A includes a component toward the base end side in the Y direction (longitudinal axis Ax direction), and the endoscope 1 is formed as a rear oblique-viewing endoscope in which a visual field range of the observation window 16 faces the base end side.

In addition, the optical axis 18A of the illumination window 18 is orthogonal to the X-Y plane. That is, the optical axis 18A is parallel to the Z direction. As described above, in a case in which the Z(+) direction side, which is the normal direction of the disposition surface 32A, is the height direction in the cover member 100 in the embodiment, the illumination window surface 101D is disposed at a position having a higher height in the height direction (normal direction of the disposition surface 32A) than the height of the observation window surface 101B, so that the illumination window 18 is located on the Z(+) direction side with respect to the observation window 16. In such a configuration, light is not directly emitted from the illumination window 18 to the observation window 16, and the occurrence of flare or the like in the observation image can be suppressed.

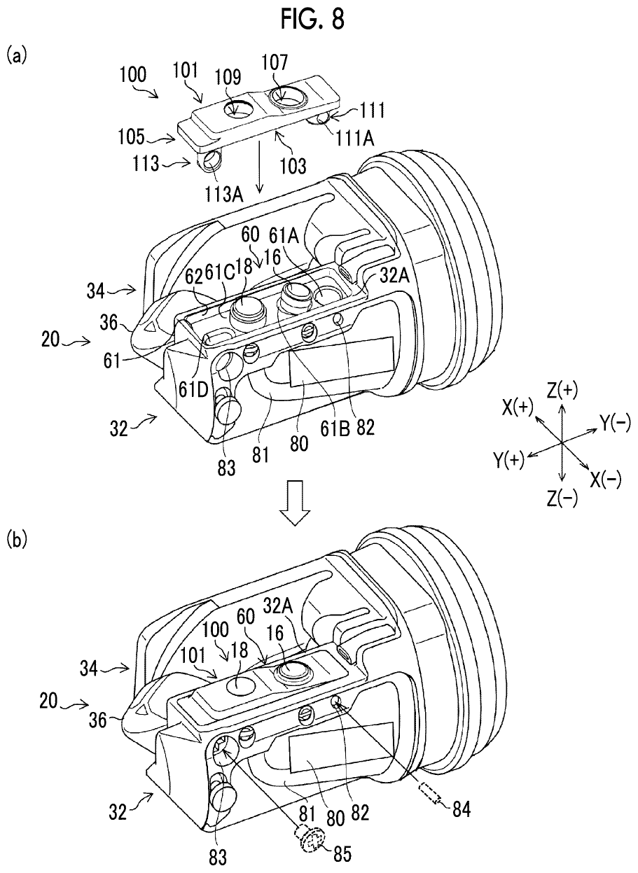

FIG. 8 is a view showing the retention of the cover member 100 and the distal end member 20. Here, (a) of FIG. 8 shows a state before the cover member 100 is disposed in the recessed portion 60. Then, (b) of FIG. 8 shows a state in which the cover member 100 is disposed in the recessed portion 60. It should be noted that, in (a) and (b) of FIG. 8, for convenience of description, a state in which the distal end member cover 33 is removed from the first wall portion 32 is shown.

As shown in (a) of FIG. 8, an imaging unit 80 and an illumination unit 81 are disposed inside the first wall portion 32. A first through-hole 82 that communicates with the first engaging hole 61A is provided inside the first wall portion 32. The first through-hole 82 has an opening portion on the X(−) direction side, and extends along the X direction. In addition, a second through-hole 83 that communicates with the second engaging hole 61D is provided. The second through-hole 83 has an opening portion on the X(−) direction side, and extends along the X direction.

In a case in which the cover member 100 is attached to the disposition surface 32A of the first wall portion 32 constituting the distal end member 20, the cover member 100 is disposed in the recessed portion 60 formed in the disposition surface 32A after the cover member 100 and the recessed portion 60 provided in the disposition surface 32A are aligned. In this case, the first protrusion portion 111 is inserted into the first engaging hole 61A, and the first through-hole 111A of the first protrusion portion 111 and the first through-hole 82 are in a state of communicating with each other. In addition, the second protrusion portion 113 is inserted into the second engaging hole 61D, and the second through-hole 113A of the second protrusion portion 113 and the second through-hole 83 are in a state of communicating with each other.

After the above-described state is obtained, a pin 84 is inserted into the first through-hole 82, and the pin 84 is caused to reach the first through-hole 111A. In addition, a screw 85 is inserted into the second through-hole 83, and the screw 85 is tightened to reach the second through-hole 113A. As a result, a state is obtained in which the cover member 100 is fixed to the disposition surface 32A of the first wall portion 32. In this case, the cover member 100 is prevented from coming off from the first wall portion 32 by the screw 85 and the pin 84. It should be noted that the pin 84 functions as a retaining portion that prevents the first protrusion portion 111 from being disengaged from the first engaging hole 61A. In addition, the screw 85 functions as a retaining portion that prevents the second protrusion portion 113 from being disengaged from the second engaging hole 61D.

In addition, as shown in (b) of FIG. 8, by disposing the cover member 100 in the recessed portion 60, most of the disposition surface 32A made of a conductive material is occupied by the cover member 100 having the insulating property, and the exposed portion of the disposition surface 32A is reduced.

It should be noted that the first engaging hole 61A and the second engaging hole 61D are examples of engaging holes constituting an engaging portion according to the embodiment of the present invention. The first protrusion portion 111 and the second protrusion portion 113 are examples of a protrusion portion constituting an engaged portion according to the embodiment of the present invention. The screw 85 and the pin 84 are examples of a retaining portion that prevents the engaged portion from being disengaged from the engaging portion according to the embodiment of the present invention.

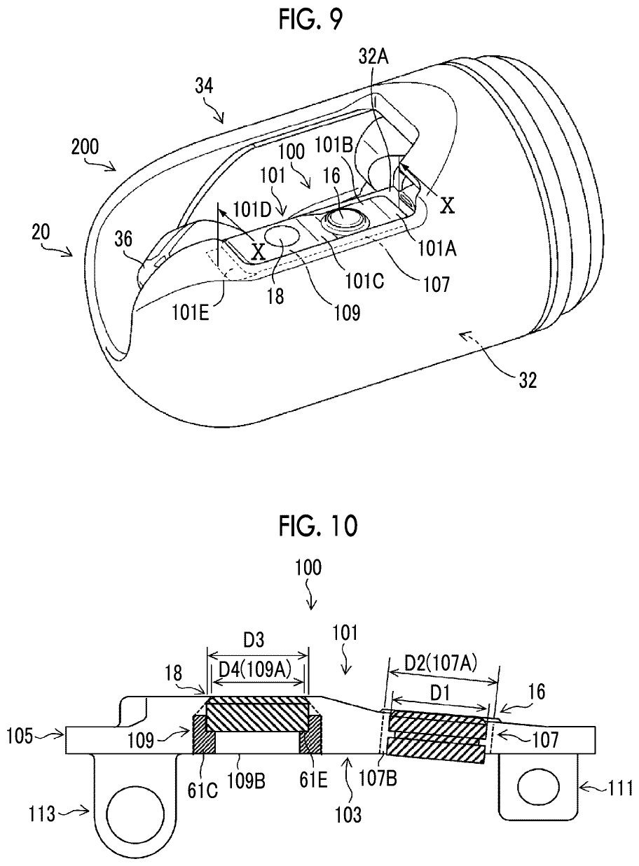

FIG. 9 is a view showing a state in which the distal end cap 200 is mounted on the distal end member 20 of (a) of FIG. 8. As shown in FIG. 9, the distal end cap 200 is mounted on the distal end member 20. In this case, the distal end side surface 101E of the upper surface 101 of the cover member 100 is covered with the distal end cap 200. By mounting the distal end cap 200 to cover a part (in the present example, the distal end side surface 101E) of a peripheral portion of the cover member 100 in this way, the insulating property of the distal end member 20 can be more reliably ensured. As a result, for example, even in a case in which the high-frequency treatment tool is used, it is possible to prevent damage to the endoscope 1 due to the generation of the spark current in advance. The distal end side surface 101E is an example of a covered portion according to the embodiment of the present invention.

It should be noted that, in the embodiment, the configuration has been described in which the distal end cap 200 covers the distal end side surface 101E that is a part of the peripheral portion of the cover member 100, but the present invention is not limited to this, and the distal end cap 200 may cover other peripheral portions of the cover member 100 other than the distal end side surface 101E. In addition, the distal end cap 200 need not cover a part of the peripheral portion of the cover member 100.

FIG. 10 is a cross-sectional view taken along a line X-X in FIG. 9. As shown in FIG. 10, the observation window 16 is disposed in the first insertion hole 107, and the illumination window 18 is disposed in the second insertion hole 109. An inner diameter D2 (inner diameters of the openings 107A and 107B) of the first insertion hole 107 is larger than an outer diameter D1 of the observation window 16. As a result, even in a case in which the cover member 100 is attached to the disposition surface 32A of the first wall portion 32, a wide visual field can be ensured without narrowing the visual field range of the observation window 16.

Meanwhile, the lower surface of the illumination window 18 is supported by the flange portion 61E. A maximum outer diameter D3 of the illumination window 18 is larger than an inner diameter D4 of the opening 109A of the second insertion hole 109. Therefore, in a case in which the cover member 100 is attached to the disposition surface 32A of the first wall portion 32, the illumination window 18 is fixed in a state of being interposed between the flange portion 61E and a tapered surface (inner surface on the upper surface 101 side) of the second insertion hole 109. As a result, the illumination window 18 is easily attached to the distal end member 20.

### Effects

As described above, according to the embodiment, in addition to the distal end cap 200 mounted on the distal end member 20 made of a conductive material, the cover member 100 having the insulating property and having the first insertion hole 107 in which the observation window 16 is disposed and the second insertion hole 109 in which the illumination window 18 is disposed is further provided on the disposition surface 32A (that is, the disposition surface 32A of the first wall portion 32 constituting the distal end member 20) on which the observation window 16 and the illumination window 18 are disposed, so that it is possible to ensure the insulating property of the distal end member 20 by suppressing the exposed portion of the conductive material.

In addition, according to the embodiment, the recessed portion 60 in which the cover member 100 is disposed is provided with the flange portion 61E that supports the lower surface of the illumination window 18, and the illumination window 18 is fixed in a state of being interposed between the cover member 100 and the flange portion 61E. As a result, the illumination window 18 is easily attached to the distal end member 20.

In addition, according to the embodiment, the cover member 100 can be easily attached to the disposition surface 32A by engaging the engaged portion (first engaging hole 61A and second engaging hole 61D) provided in the bottom surface portion 61 of the recessed portion 60 with the engaging portion (first protrusion portion 111 and second protrusion portion 113) provided in the cover member 100. In addition, since the retaining portion (screw 85 and pin 84) that prevents the engaged portion from being disengaged from the engaging portion is provided, the cover member 100 can be reliably fixed to the disposition surface 32A.

In addition, according to the embodiment, since the illumination window 18 is disposed at a position having a higher height in the height direction (normal direction of the disposition surface 32A) than the observation window 16 by the illumination window surface 101D and the observation window surface 101B provided on the cover member 100, light is not directly emitted from the illumination window 18 to the observation window 16, and the occurrence of flare or the like in the observation image can be suppressed.

Although the endoscope according to the embodiment has been described above, the present invention may be improved or modified in some ways without departing from the gist of the present invention.

### EXPLANATION OF REFERENCES

1: endoscope
2: insertion part
3: operating part
4: universal cord
5: soft portion
6: bendable portion
7: distal end portion
8: angle knob
9: angle knob
10: air/water supply button
11: suction button
12: elevating operation lever
13: treatment tool inlet port
14: air/water supply nozzle
16: observation window
16A: optical axis
18: illumination window
18A: optical axis
20: distal end member
22: elevator housing portion
24: opening
26: distal end opening
28: base portion
28A: distal end wall portion
30: flange portion
32: first wall portion
32A: disposition surface
33: distal end member cover
34: second wall portion
36: elevator
36A: guide surface
39: treatment tool outlet port
40: cover
42: lever
42A: rotation shaft
44: lever housing chamber
46: operating wire
50: guide wire fixing portion
60: recessed portion
61: bottom surface portion
61A: first engaging hole
61B: observation window hole
61C: cylindrical portion
61D: second engaging hole
61E: flange portion
62: wall surface
70: insulating ring
72: flange portion
74: cap mounting portion
76: bendable portion mounting portion
80: imaging unit
81: illumination unit
82: first through-hole
83: second through-hole
84: pin
85: screw
100: cover member
101: upper surface
101A: base end side surface
101B: observation window surface
101C: intermediate surface
101D: illumination window surface
101E: distal end side surface
101F: bank portion
103: lower surface
105: side surface
107: first insertion hole
107A: opening
107B: opening
109: second insertion hole
109A: opening
109B: opening
111: first protrusion portion
111A: first through-hole
113: second protrusion portion
113A: second through-hole
200: distal end cap
202: upper opening
204: distal end portion opening
206: base end portion opening
208: engaging groove

What is claimed is:

1. An endoscope comprising:
a distal end member that is connected to a distal end side of an insertion part and that is made of a conductive material; and
a distal end cap that is mounted on the distal end member,
wherein the distal end member has a disposition surface on which an observation window and an illumination window are disposed, and
the endoscope includes a cover member that is provided on the disposition surface, that has a first insertion hole in which the observation window is disposed and a second insertion hole in which the illumination window is disposed, and that has an insulating property.

2. The endoscope according to claim 1,
wherein the disposition surface has a recessed portion in which the cover member is disposed.

3. The endoscope according to claim 2,
wherein the recessed portion is provided with an engaging portion, and
the cover member has an engaged portion that engages with the engaging portion.

4. The endoscope according to claim 3,
wherein the engaging portion is an engaging hole that is provided in a bottom surface portion of the recessed portion, and
the engaged portion is a protrusion portion that is provided on the cover member and that is engageable with the engaging hole.

5. The endoscope according to claim 3, further comprising:
a retaining portion that prevents the engaged portion from being disengaged from the engaging portion.

6. The endoscope according to claim 2,
wherein the recessed portion has a flange portion that supports the illumination window, and
the illumination window is fixed by the cover member and the flange portion.

7. The endoscope according to claim 6,
wherein the recessed portion has a cylindrical portion that protrudes from a bottom surface portion of the recessed portion, and
the flange portion is located inside the cylindrical portion.

8. The endoscope according to claim 6,
wherein an inner diameter of the first insertion hole as viewed from an optical axis direction of the observation window is equal to or larger than an outer diameter of the observation window.

9. The endoscope according to claim 1,
wherein the cover member has a first cover surface on which the first insertion hole is formed and a second cover surface on which the second insertion hole is formed, and
the second cover surface has a higher height in a normal direction of the disposition surface than the first cover surface.

10. The endoscope according to claim 9,
wherein the cover member has an inclined surface between the first cover surface and the second cover surface.

11. The endoscope according to claim 9,
wherein a normal line of the first cover surface includes a component toward a base end side of a longitudinal axis of the insertion part.

12. The endoscope according to claim 1,
wherein an optical axis of the observation window includes a component toward a base end side of a longitudinal axis of the insertion part.

13. The endoscope according to claim 1,
wherein the cover member has a covered portion that is covered with the distal end cap in a case in which the distal end cap is mounted on the distal end member.

14. The endoscope according to claim 1,
wherein the distal end member is a side-viewing type in which the observation window and the illumination window face in a direction intersecting a longitudinal axis direction of the insertion part.
